(19) United States
(12) Patent Application Publication
(10) Pub. No.: US 2004/0242991 A1
(43) Pub. Date: Dec. 2, 2004

(54) Method for determining dosage when planning radiotherapy and/or radiosurgery procedures

(76) Inventors: Stephan Frohlich, Aschheim (DE); Werner Nowak, Munchen (DE); Claus Promberger, Munchen (DE)

Correspondence Address: Don W. Bulson, Esq., Renner, Otto, Boisselle & Sklar, P.L.L., 19th Floor, 1621 Euclid Ave., Cleveland, OH, 44115, US

(21) Appl. No.: 10/841,698

(22) Filed: May 7, 2004

Related U.S. Application Data
(60) Provisional application No. 60/479,319, filed on Jun. 18, 2003.

(30) Foreign Application Priority Data
May 8, 2003 (DE) 103 20 611.6

(51) Int. Cl. A61B 5/05

(52) U.S. Cl. 600/407

(57) ABSTRACT

A method for determining dosage when planning radiotherapy and/or radiosurgery procedures can include imaging an irradiation target area using an imaging method, which can differentiate functional and/or biologically active regions of the irradiation target area. Ascertained activity values can be allocated to individual regions of the imaged irradiation target and irradiation doses can be assigned to the regions in accordance with the activity values. A nominal dosage distribution, which can be ascertained from the irradiation dosages for the regions, can be used as an input value for treatment planning.

### RELATED APPLICATION DATA

This application claims priority of U.S. Provisional Application No. 60/479,319, filed on Jun. 18, 2003, which is incorporated herein by reference in its entirety.

### FIELD OF THE INVENTION

The invention relates to a method for determining dosage when planning radiotherapy and/or radiosurgery procedures.

### BACKGROUND OF THE INVENTION

In accordance with current prior art, irradiating particular target areas of patients, such as tumors, is planned with computer assistance and then performed on the basis of the planning, using computer-guided irradiation devices. Generally, imaging methods, such as computer tomography or nuclear spin tomography, are used to determine the outer contours of the region to be irradiated, such an outer contour in most cases being marked in on the tomographic images obtained. An irradiation target area determined in this way is generally irradiated as homogeneously as possible in accordance with conventional irradiation technology, wherein it is, in principle, unimportant whether the planning performed beforehand is performed “inversely” or conventionally.

In the case of conventional planning, a particular irradiation target is simply selected and the dosage with which the area is to be irradiated is established. Irradiation is then performed accordingly.

For inverse irradiation planning, the dosage is determined or prescribed differently. Generally, histograms (dosage-volume histograms) are used. Since in most cases perfect homogeneity cannot technically be achieved without damaging risk structures, the dosage can be prescribed, for example, in accordance with the following approach: 80% of the volume of the tumour can be irradiated with at least 90% of the prescribed dosage, 95% of the volume of the tumor can be irradiated with at least 60% of the prescribed dosage, etc.

One problem with such conventional irradiation planning is that the irradiation target area is regarded as a homogeneous mass. However, this is typically not the case, particularly with tumors. Tumors often exhibit regions of higher activity and/or aggressiveness as well as regions of low activity and/or aggressiveness. The presence of such differing regions is generally not taken into account by conventional irradiation planning, whether inverse or conventional. This may result in particular regions receiving more or less radiation than would have been necessary for an optimally successful therapy.

### SUMMARY OF THE INVENTION

In accordance with one aspect of the invention, the invention relates to a method for determining dosage when planning radiotherapy and/or radiosurgery procedures. In one embodiment, the method can enable optimized dosage planning in view of inhomogeneous activity and/or aggressiveness by treatment targets.

### BRIEF DESCRIPTION OF THE DRAWINGS

These and further features of the present invention will be apparent with reference to the following description and drawings, wherein:

FIG. 1 is a flow chart illustrating a method for determining dosage when planning radiotherapy or radiosurgery procedures in accordance with the present invention;

FIG. 2 is an exemplary functional tomographic image representation of the human head, with a visible tumor;

FIG. 3 is an enlargement of a section of the tomographic image shown in FIG. 2;

FIG. 4 is an exemplary plane representation of a nuclear medical image data set into regions of defined activity;

FIG. 5 is an exemplary dosage-volume histogram for inverse planning; and

FIGS. 6 and 7 are exemplary diagrams illustrating a linear and non-linear relationship, respectively, between activity and prescribed dosage.

### DETAILED DESCRIPTION OF THE INVENTION

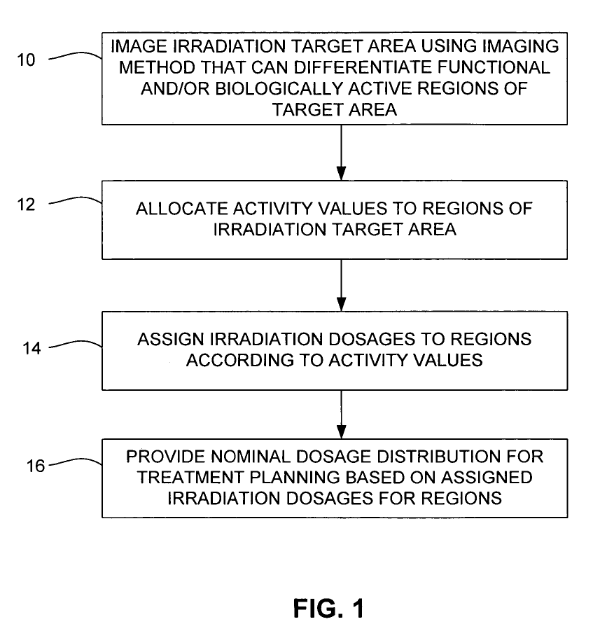

With reference to FIG. 1, one embodiment of a method for determining dosage when planning radiotherapy and/or radiosurgery procedures is provided. At step 10, an irradiation target area can be imaged. As is described more fully below, the irradiation target area can be imaged using an imaging method that can differentiate functional and/or biologically active regions of the target area. At step 12, activity values can be allocated to individual regions of the irradiation target area. At step 14, irradiation dosages can be assigned to the individual regions of the irradiation target area according to the activity values. At step 16, a nominal dosage distribution, which is ascertained from the assigned irradiation dosages for the individual regions, can be provided or otherwise used for treatment planning.

In one embodiment, information from imaging methods, which can differentiate functional and/or biologically active regions of the irradiation target area, can be utilized. Information on these “active” or “less active” regions can be used in order to also take such activity inhomogeneity into account in irradiation planning.

Imaging methods used hitherto in irradiation planning, such as computer tomography, nuclear spin tomography and x-rays more or less only provide a geometrical representation of internal structures. Other methods, for example, nuclear medical methods, such as PET and SPECT, as well as more developed methods, are increasingly gaining in importance and can be employed in conjunction with one embodiment of the invention. These imaging methods can show biology aspects. Such biology aspects can include, for example, metabolic activity, permeability of the cell wall to particular substances, and the like. In the present description, biology aspects can be characterized as those that can differentiate functional and/or biologically active regions from less active regions. From this information, the activity and/or aggressiveness in different regions of a tumor can also be detected.

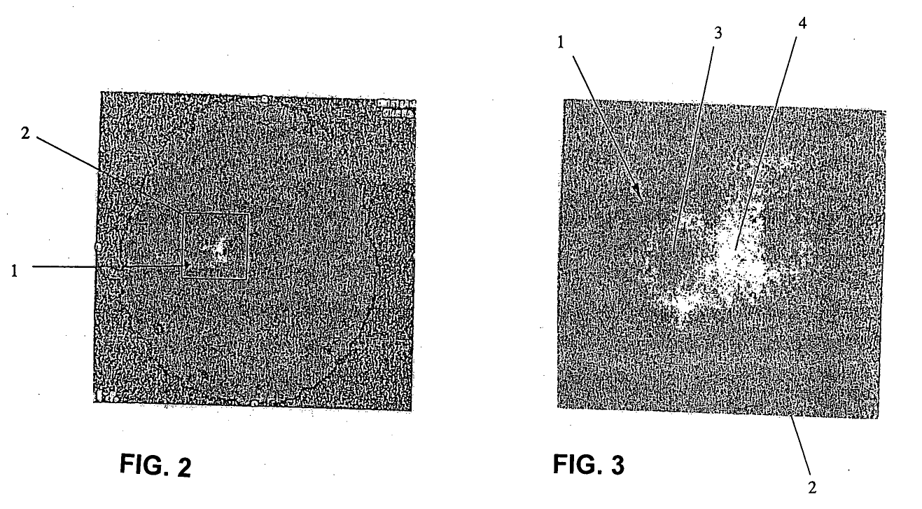

FIG. 2 shows an exemplary nuclear medical tomographic image representation of the human head, with a tumor bearing the reference numeral 1. In this case, it is a PET representation. A section 2 is shown by a square in FIG. 2, and in an enlargement in FIG. 3.

In the enlargement of the section 2 in FIG. 3, regions of differing activity can be identified in the tumor 1. A darker area is indicated by the reference numeral 3. This indicates a region of low activity and/or lower aggressiveness in the tumor. The reference numeral 4 indicates a lighter region, where the activity and/or aggressiveness is higher.

In one embodiment, it is now possible to move away from previous, maximally homogeneous irradiation and to irradiate tumours inhomogeneously, depending on the activity in the respective region. Active or “hot” regions can be heavily irradiated, while “lukewarm” regions can be irradiated more weakly.

In one embodiment, radiation dosages are assigned to the regions in accordance with the ascertained activity values. In other words, the activity in a tumor region is directly or indirectly transferred into the prescribed dosage for a corresponding tumor region.

It is to be appreciated that the invention is applicable to conventional planning, in one embodiment, and to inverse planning in another embodiment. “Inverse planning,” as used herein, broadly means that what proportion of the volume is to receive what radiation dosage is prescribed first.

Inverse planning, which regards the tumor as a homogeneous unit, can be improved to the extent that it additionally takes into account where in the tumor the more active and/or less active regions are.

In one embodiment, the activity in the tumor region can be directly transferred in a linear relationship into the prescribed dosage for the corresponding tumor region. Such a direct relationship between the dosage and activity is shown, for example, in FIG. 6. FIG. 7 shows a different arrangement, in which the activity is transferred into the prescribed dosage in a non-linear or user-defined relationship. A non-linear relationship can be established beforehand, or the relationship can also be defined manually.

This methodology provides a time-saving way of inhomogeneously, biologically prescribing the dosage. The dosage is prescribed location-specifically. In inverse planning, each individual region is targeted, rather than the complete target volume. In the clinical field, this enables less damaging irradiation, such as, for example, when a region of the tumor of less activity is near a critical (i.e., radiosensitive) structure, which does not actually need to be irradiated. It is also possible to maximize the dosage in particularly active tumor regions.

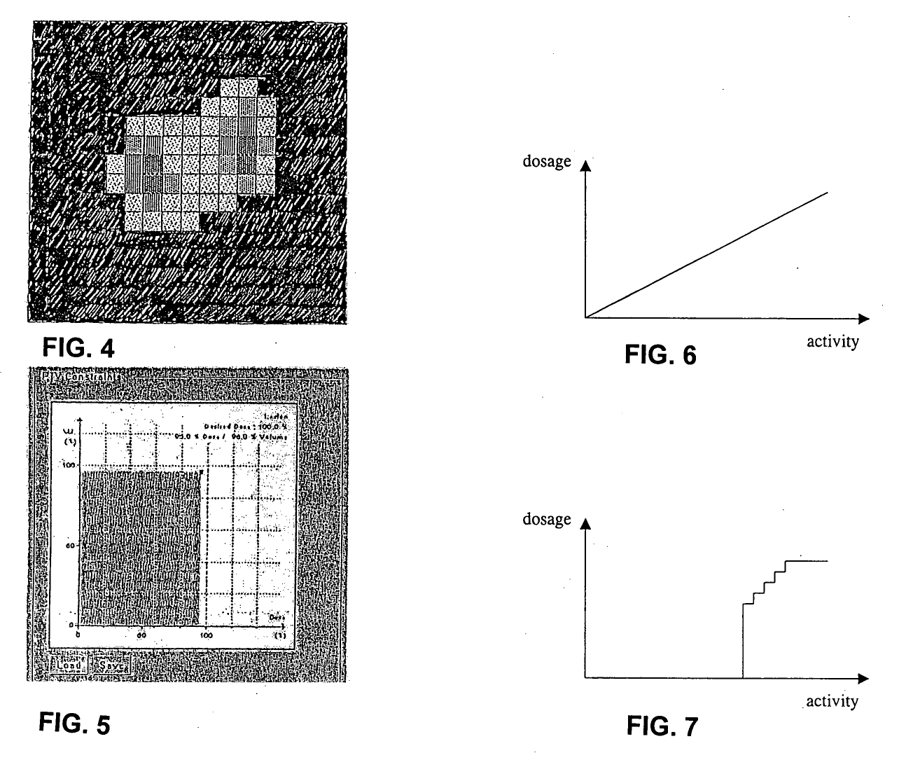

In one embodiment, regions of similar activity can be combined, such that a limited number of discrete activity levels exists. In this respect, FIG. 4 shows an example of a nuclear medical image data set divided into defined areas, with a defined dosage being allocated per area. The darker areas in FIG. 4 are indicative of a lower dosage, while the lighter grey scales tend towards higher dosages. While FIG. 4 is a plane representation of these regions, it is also possible to display such a representation three-dimensionally and utilize it three-dimensionally in planning.

It is also possible within the framework of the invention to assign an irradiation dosage only to particular user-defined regions, while the remaining regions of the irradiation target area do not receive an irradiation dosage.

In one embodiment, it is also possible to employ the more conventional imaging methods, mentioned above, as auxiliary aids, such as computer tomography, nuclear spin tomography or x-rays, which, in their basic operation, cannot differentiate functionally and/or biologically active regions. These imaging methods may provide additional information and/or allow the treatment target area to be better visualized. If the images from the different imaging techniques are registered with respect to each other (i.e., it is ensured, by navigating and/or tracking imaging, that both images show the same section) it is possible to optimally combine and utilize the information from the different imaging methods.

The irradiation dosage can be assigned to the respective region as an absolute value in accordance with the activity value, for example, 1.25 Gy. Alternatively, it is also possible to assign the irradiation dosage as a relative value, for example, as 80% of the standard dosage. The prescribed or irradiation dosage for planning can refer to the target volume(s) to be irradiated. There are also imaging methods, which, for example, display the activity of a particular brain region during a particular activity (e.g., the speech center during speaking). If this is taken into account, the irradiation dosage can refer to radiosensitive structures, which are to be irradiated as little as possible. It is of course also possible to refer the prescribed or irradiation dosage both to the target volume to be irradiated and to radiosensitive structures.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A method for determining dosage when planning radiotherapy or radiosurgery procedures, said method comprising:
imaging an irradiation target area using an imaging method that can differentiate functional and/or biologically active regions of the target area;
allocating activity values to individual regions of the irradiation target area;
assigning irradiation dosages to the individual regions in accordance with the activity values; and
providing a nominal dosage distribution for treatment planning based on the assigned irradiation dosages for the individual regions.

2. The method as set forth in claim 1, wherein the nominal dosage distribution is used as an input value for inverse treatment planning.

3. The method as set forth in claim 1, wherein the individual regions are three-dimensional regions of a defined size.

4. The method as set forth in claim 1, wherein the irradiation dosages are assigned to the individual regions in accordance with a linear relationship with the activity values.

5. The method as set forth in claim 1, wherein the irradiation dosages are assigned to the individual regions in accordance with a non-linear relationship with the activity values.

6. The method as set forth in claim 5, wherein the non-linear relationship between the activity values and the assigned irradiation dosages is defined manually.

7. The method as set forth in claim 1, said method further comprising:
combining regions of similar activity, such that a limited number of discrete activity levels exists.

8. The method as set forth in claim 1, said method further comprising:
assigning an irradiation dosage to particular user-defined regions, while the remaining regions of the irradiation target area do not receive an irradiation dosage.

9. The method as set forth in claim 8, wherein assigning an irradiation dosage to particular user-defined regions includes:
selecting the user-defined regions using image information from imaging methods that cannot differentiate functional and/or biologically active regions of the target area; and
registering image information from (i) the imaging methods that can differentiate functional and/or biologically active regions of the target area and (ii) the imaging methods that cannot differentiate functional and/or biologically active regions of the target area with respect to each other.

10. The method as set forth in claim 1, wherein the irradiation dosage is assigned as an absolute value.

11. The method as set forth in claim 1, wherein the irradiation dosage is assigned as a percentage of a standard dosage.

12. The method as set forth in claim 1, said method further comprising:
assigning the irradiation dosage in accordance with at least one of (i) areas desired to be highly irradiated and (ii) areas desired to be weakly irradiated.
